(19) United States
(12) Patent Application Publication
(10) Pub. No.: US 2010/0034760 A1
(43) Pub. Date: Feb. 11, 2010

(54) Method of Increasing the Tanning Effect of Self-tanning Substances

(75) Inventors: Thomas Rudolph, Darmstadt (DE); Philipp Buehle, Darmstadt (DE); Hansjuergen Driller, Gross-Umstadt (DE); Herwig Buchholz, Frankfurt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 12/518,585

(22) PCT Filed: Nov. 20, 2007
(86) PCT No.: PCT/EP2007/010020

§ 371 Date: Jun. 10, 2009

(30) Foreign Application Priority Data
Dec. 11, 2006 (DE) 10 2006 058 237.3

(51) Int. Cl. A61K 8/35; A61K 8/00; A61Q 17/04; A61K 8/23

(52) U.S. Cl. 424/59

(57) ABSTRACT

The invention relates to a method for enhancing the tanning action of at least one self-tanner substance by reducing or eliminating the presence of oxygen.

The invention relates to a method for enhancing the tanning action of at least one self-tanner substance by reducing or eliminating the presence of oxygen.

The trend away from fashionable paleness to “healthy, sportily brown skin” has been uninterrupted for years. In order to achieve this, people expose their skin to sunlight since this causes pigmentation due to melanin formation. However, the UV radiation of sunlight also has a damaging effect on the skin. Besides acute damage (sunburn), long-term damage occurs, on excessive irradiation with light from the UVB region (wavelength 280-320 nm), such as, for example, an increased risk of developing skin cancer. In addition, excessive exposure to UVB and UVA radiation (wavelength: 320-400 nm) results in a weakening of the elastic and collagenic fibres of the connective tissue. This results in numerous phototoxic and photoallergic reactions and results in premature skin ageing.

Natural protection against the adverse consequences of sunlight is offered by tanning (pigmentation) of the skin. In its lowermost layer, the basal layer, the epidermis contains individual pigment-forming cells, the melanocytes, in addition to the basal cells. UV light stimulates the production of melanin in these cells, which is transported into the keratinocytes, where it becomes visible as a brown skin colour.

This pigment formation starting from the amino acid tyrosine is initiated predominantly by UVB radiation and is referred to as “indirect pigmentation”. Its development proceeds over a number of days; the resultant suntan lasts for a few weeks.

In the case of “direct pigmentation”, which commences with solar irradiation, predominantly colourless melanin precursors are oxidised by UVA radiation to dark-coloured melanin. Since this oxidation is reversible, it results in skin tanning which only lasts briefly.

Artificial tanning of the skin can be produced externally with the aid of make-up and orally by taking carotenoids.

Much more popular, however, is artificial tanning of the skin which can be achieved by application of so-called self-tanners. These compounds have, as chemical structural feature, keto or aldehyde groups in the vicinity of alcohol functions. These ketols or aldols belong predominantly to the sugars class of substances. A self-tanning substance which is employed particularly frequently is 1,3-dihydroxyacetone (DHA).

The compounds can be reacted with the proteins and amino acids of the horny layer of the skin in the sense of a Maillard reaction, where a reaction route which has not yet been fully clarified results in polymers which provide the skin with a brownish hue. This reaction is complete after about 4 to 6 hours. The tan achieved in this way cannot be washed off and is only removed with the normal skin desquamation.

The self-tanner substances are usually sprayed or applied manually to the skin as a solution or emulsion. However, tanning of the skin generally only takes place after a delay through very slow reaction of the self-tanner with the proteins of the skin, as described above. It is all the more desired, therefore, in particular from the user point of view, that the tanning per application of the self-tanner substance is enhanced and tanning is achieved more quickly overall.

The object of the present invention was accordingly to find a method for enhancing the tanning action of self-tanner substances.

It has now been found, surprisingly, that tanning enhancement occurs to a large extent if the air present during application or the permanent air—or the oxygen present therein—is reduced or eliminated. In addition, the tanning of the skin generally becomes more uniform and lasts longer. Depending on which method is used for reducing the oxygen, an acceleration of the tanning action may also occur, i.e. in other words the tanning occurs earlier in the period observed. The term “tanning enhancement” is also used in this sense in accordance with the invention.

The invention therefore relates firstly to a method for enhancing the tanning action of at least one self-tanner substance by reducing or eliminating the presence of oxygen.

Suitable measures for the reduction of air or oxygen exposure are, for example, the type of application, the composition of the preparation comprising the at least one self-tanner substance or the composition of the environment, based on physical, chemical, biochemical or microbiological effects.

For the purposes of the invention, the type of application includes the time and manner of the application, as described in detail below, or the type and manner of the application of the self-tanner substance or the treatment of the area to be tanned.

For the purposes of the invention, the oxygen may be reduced or eliminated in the form of pure oxygen, but also in mixtures with other gases, for example as an oxygen/carbon dioxide mixture or also generally atmospheric oxygen.

The invention therefore furthermore relates to a method for enhancing the tanning action of at least one self-tanner substance, where the reduction or elimination of the oxygen content is achieved during application of the at least one self-tanner substance.

Exclusion of oxygen or a reduction in the concentration of oxygen in the upper skin layers during application can be carried out, for example, if the application of the preparation comprising at least one self-tanner substance is carried out in a tanning reactor under a protective gas. A tanning reactor is taken to mean, for example, a tanning cabin in which the cosmetic preparation comprising the at least one self-tanner substance is applied to the skin, for example, through nozzles, a tanning shower, a tanning bath or tanning systems, such as, for example, the airbrush tanning system from Beauty-Form, where the tanning system produces a spray mist of a tanning lotion, which is applied to the skin.

In a further and preferred variant of the invention, the reduction or elimination of the oxygen content can be achieved by pretreatment of the skin to which the at least one self-tanner substance is applied.

This pretreatment can be carried out, for example, by application of a formulation comprising at least one oxygen-withdrawing or oxygen-binding substance and/or at least one oxygen-consuming biochemical or microbiological component and/or at least one antioxidant.

For the purposes of the present invention, the term component is used synonymously with substance.

For the purposes of the present invention, the term kit is used synonymously with set.

For the purposes of the present invention, composition or preparation is also used synonymously alongside the term formulation.

For the purposes of the present invention, self-tanning substance is also used alongside the term self-tanner substance.

For the purposes of the present invention, dihydroxyacetone or the abbreviation DHA is also used synonymously alongside 1,3-dihydroxyacetone. Inertised preparation means that measures to reduce or eliminate the oxygen content of the preparation have been taken for a preparation. A suitable measure is degassing, where the oxygen content in the aqueous component of the preparation, as described above, is less than or equal to 10 mg/l or preferably in the range between 0.1 and 7 mg/l. Naturally, the oxygen content in the oil phase of the preparation may be or is also reduced by this measure.

Suitable oxygen-withdrawing or oxygen-binding substances are, for example, alkali metal, alkaline-earth metal or ammonium sulfites, alkali metal, alkaline-earth metal or ammonium hydrogensulfites, alkali metal, alkaline-earth metal or ammonium bisulfites, alkali metal, alkaline-earth metal or ammonium polysulfites or dialkylhydroxylamines.

An alkyl group is taken to mean, for example, an alkyl group having 1, 2, 3, 4, 5 or 6 C atoms, for example methyl, ethyl, isopropyl, n-propyl, isobutyl, n-butyl, tert-butyl, n-pentyl or n-hexyl.

Dialkylhydroxylamines are, for example, dimethylhydroxylamine, methylethylhydroxylamine, diethylhydroxylamine, dipropylhydroxylamine, dibutylhydroxylamine, dipentylhydroxylamine or dihexylhydroxylamine, where diethylhydroxylamine can preferably be employed in accordance with the invention.

Preferably suitable in accordance with the invention are sodium bisulfite, sodium hydrogensulfite, sodium sulfite, potassium hydrogensulfite, potassium sulfite, ammonium hydrogensulfite, ammonium sulfite, magnesium hydrogensulfite or magnesium sulfite, where sodium bisulfite, sodium sulfite or potassium sulfite can particularly preferably be used or sodium sulfite can very particularly preferably be used.

The oxygen-binding or oxygen-withdrawing substances, as described above, are typically employed in accordance with the invention in amounts of 0.01 to 20% by weight, preferably in amounts of 0.05% by weight to 10% by weight, in the formulation for pretreatment. The amount data are based on the total amount of the formulation for pretreatment. The person skilled in the art is presented with absolutely no difficulties here in selecting the amounts correspondingly depending on the intended action of the preparation.

Suitable oxygen-consuming biochemical or microbiological components are, for example, superoxide dismutase, peroxidase and/or catalase, in each case as the enzyme or isoenzyme.

The synergistic antioxidative action of superoxide dismutase and peroxidase is known from Int. J. Cos. Sci 2000, Lods, 85ff. The oxygen-degrading activity of an isoenzyme of superoxide dismutase is known from WO 2005/017134.

Superoxide dismutase, peroxidase and/or catalase can be used both as pure substance or extract. Usual use concentrations are between 0.1% by weight and 10% by weight, preferably 2% by weight, based on the total amount of the preparation.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to pmol/kg), and also (metal) chelating agents (for example α-hydroxyfatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the general formula A or B

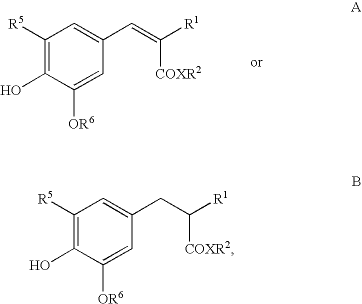

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes linear or branched alkyl having 1 to 8 C atoms or linear or branched alkoxy having 1 to 8 C atoms, and
R⁶ denotes linear or branched alkyl having 1 to 8 C atoms, preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-dimethoxybenzyl)malonate (for example RonaCare® AP).

Mixtures of antioxidants are likewise suitable for use according to the invention for pretreatment in the cosmetic preparations. Known and commercial mixtures are, for example, mixtures comprising, as active compounds, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004).

The antioxidants, as described above, are typically employed in accordance with the invention in amounts of 0.01 to 20% by weight, preferably in amounts of 0.05% by weight to 10% by weight, based on total amount in the formulation for pre-treatment. The person skilled in the art is presented with absolutely no difficulties here in selecting the amounts correspondingly depending on the intended action of the preparation.

The cosmetic preparation comprising at least one oxygen-withdrawing or oxygen-binding substance and/or at least one oxygen-consuming biochemical or microbiological component and/or at least one antioxidant should be applied rapidly to the area later to be treated with the formulation comprising at least one self-tanner substance. The application is preferably carried out over the course of 1 to 15 minutes, naturally depending on the size of the area, with an exposure time of up to one hour. The formulation comprising the at least one self-tanner substance should then be applied.

The invention furthermore relates to a kit comprising at least one preparation comprising at least one oxygen-binding or oxygen-withdrawing substance, as described above, and/or at least one oxygen-consuming biochemical or microbiological component, as described above, and/or at least one antioxidant, as described above,

and at least one preparation comprising at least one self-tanner substance. Preparations comprising at least one antioxidant, as described above, are known to the expert world. In the assembly as a kit, however, a preparation comprising an antioxidant, a preparation comprising an oxygen-binding or oxygen-withdrawing substance, a preparation comprising the oxygen-consuming biochemical or microbiological component can also be combined with a preparation comprising at least one self-tanner substance. The kit may also consist of the said preparations.

The kit can be supplied in packaging or also separately, where the individual constituents of the kit, i.e. the preparations, as described above, are present or consist thereof.

In a further and preferred variant of the invention, the reduction or elimination of the oxygen content can take place after application of the at least one self-tanner substance.

A suitable measure for this purpose is, for example, covering of the area treated with a preparation comprising at least one self-tanner substance by cloths, films or other materials, where, in particular, the material used should have low oxygen permeability. Low oxygen permeability is defined as a value of less than 1000 cm³/(m²*bar*d), preferably less than 100 cm³/(m²*bar*d), particularly preferably less than 50 cm³/(m²*bar*d). The material used is ideally permeable to water.

The gas permeability can be measured via the carrier-gas method (DIN 53380-3 or DIN 53380-S). The sample is installed in a permeation cell in such a way that it forms the barrier between two separated chambers. The test gas, here oxygen, flows through one measurement chamber under test pressure. A carrier gas, for example nitrogen, which transports the test gas permeating through the sample to the sensor, flows through the other chamber. The films ideally have the size of a DIN A4 sheet. In general, the test is carried out on three independent samples per permeation direction. The test temperatures are generally between −50° C. and 50° C. at pressures up to 100 bar.

In accordance with the invention, preference is given to the use of films made from plastics or composite films, for example polyethylene or polypropylene films, PVC, PVDC, PA, PET, EVOH films, but also special composite films, such as, for example, Escal™ film from Mitsubishi (PP/ceramic-coated PVA/PE having an oxygen permeability of 0.05 cm³/(m²*atm*d)), or aluminium composite films (polyester/aluminium/PP or PET/AI/PE, oxygen permeability <0.01 cm³/(m²*atm*d)) can also be employed.

Abbreviations used are:

PE polyethylene
PP polypropylene
PVC polyvinyl chloride
PVDC polyvinylidene chloride
PA polyamide
PET polyethylene terephthalate
EVOH ethylene-vinyl alcohol
PVOH or PA polyvinyl alcohol
Al aluminium.

The area treated with the at least one self-tanner substance should be covered with the correspondingly selected material, as described above, for at least 10 minutes or longer. It should be noted here that the measure taken, as described above, reduces the concentration of oxygen above the skin and in the upper skin layers, or can reduce the presence of oxygen to a partial pressure less than the atmospheric partial pressure of oxygen. The atmospheric partial pressure of oxygen is physically defined as 159.21 mmHg at sea level.

The invention therefore furthermore relates to a kit comprising a formulation comprising at least one self-tanner substance and a film whose oxygen permeability has a value of less than 1000 cm³/(m²*bar*d).

In a further variant of the invention, the reduction or elimination of the oxygen content can be achieved through the type of formulation comprising the at least one self-tanner substance.

One possibility here is to prepare the formulation with a reduction in the oxygen content. For example, the individual components of the preparation which comprises the at least one self-tanner substance can be degassed or inertised and subsequently mixed with one another. However, it is also possible firstly to prepare the preparation by conventional methods known to the person skilled in the art and then to degas or inertise it. It should be ensured during degassing or inertisation that the content of oxygen in the aqueous component of the preparation, as described above, is less than or equal to 10 mg/l or preferably in the range between 0.1 and 7 mg/l. The oxygen content in the aqueous solution is determined using the titrimetric oxygen test from Merck KGaA, Darmstadt, Germany [1.11107.0001]. The dissolved oxygen oxidises manganese(II) ions in alkaline solution to manganese(IV) oxide hydrates—so-called “oxygen fixing” takes place. In strongly acidic solution, manganese(III) ions form therefrom, which oxidise iodide ions to iodine. The resultant iodine is titrated against starch as indicator with the aid of a sodium thiosulfate solution until decolorisation is complete. The oxygen concentration arises from the consumption of titration solution. This test is based on the Winkler iodometric determination. The determination limit is 0.1 mg/l of oxygen in the aqueous solution. An experimental description of this test method is given in a corresponding example in the example part.

Degassing or inertisation of the preparation, as described above, is carried out, for example, by passing a stream of inert gas through liquid individual components or liquid components of the preparation. The stream of inert gas ideally consists of nitrogen, argon, other inert gases or mixtures thereof.

The invention therefore furthermore relates to an inertised preparation comprising at least one self-tanner substance. The oxygen content in the aqueous component of the preparation is preferably less than or equal to 10 mg/l. The preparation of an inertised preparation of this type comprising at least one self-tanner substance is described above.

A further possibility for reducing or eliminating the oxygen content through the type of formulation comprising the at least one self-tanner substance is, for example, the addition of at least one oxygen-binding component and/or at least one oxygen-consuming biochemical or microbiological component to this preparation.

The oxygen-binding or oxygen-withdrawing substances or components which can be used have previously been described for the preparation for pretreatment of the skin. The admixing of sodium sulfite or diethylhydroxylamine with the self-tanner preparation is regarded as very particularly preferred here too.

The oxygen-binding or oxygen-withdrawing compounds are typically employed in accordance with the invention in amounts of 0.01 to 20% by weight, preferably in amounts of 0.05% by weight to 10% by weight, based on the total amount of the preparation. The person skilled in the art is presented with absolutely no difficulties here in selecting the amounts correspondingly depending on the intended action of the preparation.

The oxygen-consuming biochemical or microbiological components which can be used have previously been described for the preparation for pre-treatment of the skin. The admixing of superoxide dismutase and/or peroxidase with the self-tanner preparation is regarded as very particularly preferred here. The enzymes or isoenzymes can be used both as pure substance or extract. Usual use concentrations are between 0.1% by weight and 10% by weight, preferably 2% by weight, based on the total amount of the preparation.

The invention therefore furthermore also relates to a preparation comprising at least one self-tanner substance and at least one oxygen-consuming biochemical or microbiological component, as described above.

The enhancement according to the invention of the tanning action of the at least one self-tanner substance can of course also be achieved by any desired combination of the measures outlined above. An example of such a combination is the successive covering of the skin area treated with an inertised preparation comprising the at least one self-tanner substance.

The method described above for enhancing the tanning action and the requisite measures that can be taken generally applies or apply to all known self-tanning substances or all cosmetic or dermatological self-tanning formulations comprising at least one self-tanner substance, mixtures of self-tanner substances or combinations of self-tanner substances with action enhancers, for example with flavonoids or further active substances, auxiliaries or additives.

Self-tanning substances which can be employed are, inter alia:

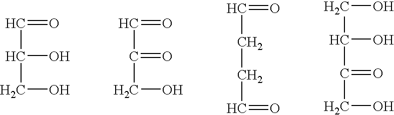

glycerolaldehyde hydroxymethylglyoxal γ-dialdehyde erythrulose (glyceraldehyde)

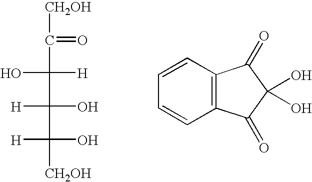

6-aldo-D-fructose ninhydrin,
furthermore 5-hydroxy-1,4-naphthoquinone (juglone), which can be extracted from the shells of fresh walnuts,

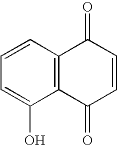

5-hydroxy-1,4-naphthoquinone (juglone),
and 2-hydroxy-1,4-naphthoquinone (lawsone), which occurs in henna leaves,

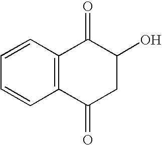

2-hydroxy-1,4-naphthoquinone (lawsone), 1,3-dihydroxyacetone (DHA), dihydroxyacetone phosphate, glyceraldehyde phosphate and erythrose.

The following trioses and tetroses are preferably used:

1,3-dihydroxyacetone (DHA), glyceraldehyde, dihydroxyacetone phosphate, glyceraldehyde phosphate, erythrose and 1,3,4-trihydroxy-2-butanone(erythrulose).

The tanning action of 1,3-dihydroxyacetone and erythrulose is enhanced to a particular extent by the said measures. The tanning action of 1,3-dihydroxyacetone is enhanced to a very particular extent by the said measures.

The at least one self-tanner substance in the corresponding compositions or preparations is typically employed in accordance with the invention in amounts of 0.01 to 20% by weight, preferably in amounts of 0.05% by weight to 10% by weight, based on the total amount of the preparation. The person skilled in the art is presented with absolutely no difficulties here in selecting the amounts correspondingly depending on the intended action of the preparation. In the case of a mixture of self-tanner substances, the percent by weight ratio is preferably between 1:10 and 10:1. A preferred mixture of self-tanner substances is the mixture of DHA and erythrulose. Mixing ratios in percent by weight of DHA:erythrulose of 2:1 and 3:1, for example, are employed.

The compositions or formulations indicated here comprising the at least one self-tanner substance or also comprising the at least one oxygen-binding or oxygen-withdrawing and/or the at least one oxygen-consuming biochemical or microbiological component and/or at least one antioxidant, the kit consisting of or comprising preparations or also the inertised preparation according to the invention are usually preparations which can be used topically, for example cosmetic, pharmaceutical or dermatological formulations. In this case, the preparations comprise a cosmetically, pharmaceutically or dermatologically suitable vehicle and, depending on the desired property profile, optionally further suitable ingredients. The topical preparations are preferably employed as cosmetic or dermatological preparation, particularly preferably as cosmetic preparation.

“Can be used topically” means suitable for a local form, in particular a form which can be applied to the surface.

The compositions used in accordance with the invention or the preparations according to the invention may comprise, as part of the kit, vitamins, inorganic or organic UV filters as further ingredients. Particular preference is given to UV filters whose physiological acceptability has already been demonstrated. There are many proven substances which are known from the specialist literature, both for UVA and also UVB filters.

The preparations comprising the at least one self-tanner substance or also the preparation for pretreatment may in addition comprise further anti-ageing active compounds, anticellulite active compounds or conventional skin-protecting or skin-care active compounds. Skin-protecting or skin-care active compounds may in principle be all active compounds known to the person skilled in the art.

The compositions or preparations described above may therefore preferably comprise, as further ingredients, vitamins and/or vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably retinol, nicotinamide, vitamin A palmitate, vitamin C and derivatives thereof, DL-α-tocopherol, tocopherol E acetate, nicotinic acid, pantothenic acid and biotin, very particularly preferably retinol and nicotinamide. Vitamins are usually employed here with compounds of the formula I in percent by weight ratios in the range from 1000:1 to 1:1000, preferably in percent by weight ratios of 100:1 to 1:100.

The compositions or preparations described above may therefore preferably comprise, as further ingredients, UV filters as described above, for example

benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007), ethoxylated ethy 4-aminobenzoate (for example Uvinul® P25),
phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid,
and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-ylmethanesulfonic acid and salts thereof (for example Mexoryl® SX),
2,4,6-trianilino-(p-carbo-2′-ethylhexyl-1′-oxy)-1,3,5-triazine (for example Uvinul® T 150),
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

These organic UV filters are generally incorporated into cosmetic formulations in an amount of 0.5 to 10 percent by weight, preferably 1-8% by weight. The amount data are based on the total amount of the formulation.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (INCI: Drometrizole Trisiloxane, for example Mexoryl® XL),
α-(trimethylsilyl)-ω-[trimethylsilyl)oxy]poly[oxy(dimethyl[and approximately 6% of methyl[2-[p-[2,2-bis(ethoxycarbonyl)vinyl]phenoxy]-1-methyleneethyl] and approximately 1.5% of methyl[3-[p-[2,2-bis(ethoxycarbonyl)vinyl)phenoxy)propenyl) and 0.1 to 0.4% of (methylhydrogen]-silylene]] (n≈60) (CAS No. 207 574-74-1) (INCI: Polysilicone-15, for example Parsol® SLX),
2,2′-methylenebis(6-(2H-benzotriazol-2-yl)-4-(1,1,3,3-tetramethylbutyl)phenol) (CAS No. 103 597-45-1) (INCI: Methylene Bis-Benzotriazolyl Tetramethylbutylphenol, for example Tinosorb® M),
2,2′-(1,4-phenylene)bis(1H-benzimidazole-4,6-disulfonic acid, monosodium salt) (CAS No. 180 898-37-7),
2,4-bis{[4-(2-ethylhexyloxy)-2-hydroxylphenyl}-6-(4-methoxyphenyl)-1,3,5-triazine (CAS No. 103 597-45-, 187 393-00-6) (INCI: Bis-Ethylhexyloxyphenol Methoxyphenyl Triazine, for example Tinosorb® S) or
2-ethyl hexyl 4,4′-[(6-[4-((1,1-dimethylethyl)aminocarbonyl)phenylamino]-1,3,5-triazine-2,4-diyl)diimino]bis(benzoate) (INCI: Diethylhexyl Butamido Triazone, for example Uvasorbo HEB).

Organic UV filters are generally incorporated into cosmetic formulations in an amount of 0.5 to 20 percent by weight, preferably 1-15% by weight. The amount data are based on the total amount of the formulation.

Conceivable inorganic UV filters are those from the group of the titanium dioxides, such as, for example, coated titanium dioxide (for example Eusolex® T-2000, Eusolex® T-AQUA, Eusolex® T-AVO), zinc oxides (for example Sachtotec®), iron oxides or also cerium oxides. These inorganic UV filters are generally incorporated into cosmetic preparations in an amount of 0.5 to 20 percent by weight, preferably 2-10% by weight. The amount data are based on the total amount of the formulation.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous preparations. In addition, the oily impression on application of the preparation comprising hydrophobic UV filters, which is frequently regarded as unpleasant, is suppressed.
Certain UV filters, in particular dibenzoylmethane derivatives, exhibit only reduced photostability in cosmetic preparations. Encapsulation of these filters or compounds which impair the photostability of these filters, such as, for example, cinnamic acid derivatives, enables the photostability of the entire preparation to be increased.
Skin penetration by organic UV filters and the associated potential for irritation on direct application to the human skin is repeatedly being discussed in the literature. The encapsulation of the corresponding substances which is proposed here suppresses this effect.
In general, encapsulation of individual UV filters or other ingredients enables problems with the preparation caused by the interaction of individual preparation constituents with one another, such as crystallisation processes, precipitation and agglomerate formation, to be avoided since the interaction is suppressed.

It is therefore preferred for one or more of the above-mentioned UV filters to be in encapsulated form. It is advantageous here for the capsules to be so small that they are invisible to the naked eye. In order to achieve the above-mentioned effects, it is furthermore necessary for the capsules to be sufficiently stable and the encapsulated active compound (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules particularly preferably to be employed in accordance with the invention have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules in the preparations are preferably present in amounts which ensure that the encapsulated UV filters are present in the preparation in the above-indicated amounts.

The compositions or preparations described above may therefore preferably comprise, as further ingredients, anti-ageing active compounds, anti-cellulite active compounds or conventional skin-protecting or skin-care active compounds.

Particularly preferred anti-ageing active compounds are pyrimidinecarboxylic acids, aryl oximes, bioflavonoids, bioflavonoid-containing extracts, chromones or retinoids.

Pyrimidinecarboxylic acids occur in halophilic microorganisms and play a role in osmoregulation of these organisms (E. A. Galinski et al., Eur. J. Biochem., 149 (1985) pages 135-139). Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacteria cells. Pharmaceutical products, such as non-glycosylated, pharmaceutically active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic preparations, such as powders, soaps, surfactant-containing cleansing products, lipsticks, rouge, make-up, care creams and sunscreen preparations.

Preference is given here to the use of a pyrimidinecarboxylic acid of the following formula:

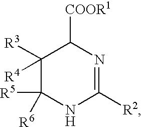

in which R¹ is a radical H or C₁₋₈-alkyl, R² is a radical H or C₁₋₄-alkyl, and R³, R⁴, R⁵ and R⁶ are each, independently of one another, a radical from the group H, OH, NH₂ and C₁₋₄-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). The preparations according to the invention preferably comprise pyrimidinecarboxylic acids of this type in amounts of up to 15% by weight. The pyrimidinecarboxylic acids are preferably employed here in percent by weight ratios of 100:1 to 1:100 with respect to the compounds of the formula I, with percent by weight ratios in the range 1:10 to 10:1 being particularly preferred.

Of the aryl oximes, preference is given to the use of 2-hydroxy-5-methyllaurophenone oxime, which is also known as HMLO, LPO or F5. Its suitability for use in cosmetic compositions is known, for example, from DE-A-41 16 123. Preparations which comprise 2-hydroxy-5-methyllaurophenone oxime are accordingly suitable for the treatment of skin diseases which are associated with inflammation. The preparations here preferably comprise 0.01% to 10% by weight of the aryl oxime, it being particularly preferred for the preparation to comprise 0.05 to 5% by weight of aryl oxime. The amount data are based on the total amount of the formulation.

Known bioflavonoids are, for example, troxerutin, tiliroside, α-glucosylrutin, rutin or isoquercetin, where the said choice is not intended to have a restrictive effect.

Bioflavonoid-containing extracts are, for example, gingko biloba or emblica.

Known anti-ageing substances are also chromones, as described, for example, in EP 1508327, or retinoids, for example retinol (vitamin A), retinoic acid, retinaldehyde or also synthetically modified compounds of vitamin A.

The chromones and retinoids described are simultaneously also effective anticellulite active compounds. A likewise known anticellulite active compound is caffeine.

The compositions may include or comprise, essentially consist of or consist of the said necessary or optional constituents or ingredients. All compounds or components which can be used in the preparations are either known and commercially available or can be synthesised by known processes.

The preparations described above are suitable for external use, for example in the form of a cream, lotion, gel or as a solution which can be sprayed onto the skin.

Examples of application forms of these preparations which may be mentioned are: solutions, suspensions, emulsions, PIT emulsions, pastes, ointments, gels, creams, lotions, powders, surfactant-containing cleansing preparations, oils, aerosols and sprays. Examples of other application forms are shower preparations. Any desired customary vehicles, auxiliaries and, if desired, further active compounds may be added to the preparation.

Preferred auxiliaries originate from the group of the preservatives, stabilisers, solubilisers or odour improvers.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, dimethyl capramide, dimethyl isosorbide, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

The preferred preparation forms include, in particular, emulsions.

Emulsions are advantageous and comprise, for example, the said fats, oils, waxes and other lipids, as well as water and an emulsifier, as usually used for a preparation of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic lipids, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

The oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of the branched and unbranched hydrocarbons and hydrocarbon waxes, silicone oils, dialkyl ethers, the group of the saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18, C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed. It may also be advantageous to employ waxes, for example cetyl palmitate, as the only lipid component of the oil phase.

The oil phase is advantageously selected from the group 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicaprylyl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used is advantageously cyclomethicone (octamethylcyclotetrasiloxane). However, it is also advantageous to use other silicone oils, for example hexamethylcyclotrisiloxane, polydimethylsiloxane, poly(methylphenylsiloxane).

Also particularly advantageous are mixtures of cyclomethicone and isotridecyl isononanoate, of cyclomethicone and 2-ethylhexyl isostearate.

The aqueous phase of the preparations described above optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions are advantageous and comprise, for example, the said fats, oils, waxes and other lipids, as well as water and an emulsifier, as usually used for a formulation of this type.

In a preferred embodiment, the preparations described above comprise hydrophilic surfactants.

The hydrophilic surfactants are preferably selected from the group of the alkylglucosides, acyl lactylates, betaines and coconut amphoacetates.

The alkylglucosides are themselves advantageously selected from the group of the alkylglucosides which are distinguished by the structural formula

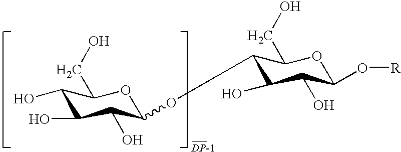

where R represents a branched or unbranched alkyl radical having 4 to 24 carbon atoms, and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = \sum \frac{{p}_{i}}{100} \cdot i$$

in which p₁, p₂, p₃ . . . pᵢ represent the proportion of mono-, di-, tri- . . . i-fold glucosylated products in percent by weight. Advantageous in accordance with the invention is the selection of products having degrees of glucosylation of 1-2, particularly advantageously of 1.1 to 1.5, very particularly advantageously of 1.2-1.4, in particular of 1.3.

The value DP takes into account the fact that alkylglucosides generally, as a consequence of their preparation, represent mixtures of mono- and oligoglucosides. A relatively high content of monoglucosides, typically in the order of 40-70% by weight, is advantageous in accordance with the invention.

Alkylglucosides advantageously used are selected from the group octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and auxiliaries or mixtures which are distinguished by an effective content of the active compounds used in accordance with the invention, for example Plantaren® 1200 (Henkel KGaA), Oramix® NS 10 (Seppic).

The acyllactylates are themselves advantageously selected from the group of the substances which are distinguished by the structural formula

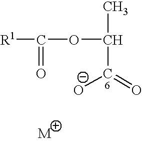

where R¹ denotes a branched or unbranched alkyl radical having 1 to 30 carbon atoms, and M⁺ is selected from the group of the alkali metal ions and the group of ammonium ions which are substituted by one or more alkyl and/or one or more hydroxyalkyl radicals, or corresponds to half an equivalent of an alkaline-earth metal ion.

For example, sodium isostearyl lactylate, for example the product Pathionic® ISL from the American Ingredients Company, is advantageous. The betaines are advantageously selected from the group of the substances which are distinguished by the structural formula

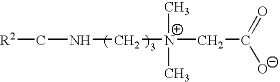

where R² denotes a branched or unbranched alkyl radical having 1 to 30 carbon atoms.

R² particularly advantageously denotes a branched or unbranched alkyl radical having 6 to 12 carbon atoms.

For example, capramidopropylbetaine, for example the product Tego® Betain 810 from Th. Goldschmidt AG, is advantageous.

A coconut amphoacetate which is advantageous in accordance with the invention is, for example, sodium coconut amphoacetate, as available from Miranol Chemical Corp. under the name Miranol® Ultra C32.

The preparations described above are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological preparations described above are applied to the skin in an adequate amount in the usual manner for cosmetics.

The cosmetic and dermatological preparations can exist in various forms. Thus, they can be, for example, a solution, a water-free preparation, an emulsion or microemulsion of the water-in-oil (W/O) type or of the oil-inwater (O/W) type, a multiple emulsion, for example of the water-in-oil-inwater (W/O/W) type, a gel, a solid stick, an ointment or an aerosol. It is also advantageous to administer ectoine in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

Co-emulsifiers which are advantageously selected are, for example, O/W emulsifiers, principally from the group of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or in the case of isoalkyl derivatives, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Optional W/O emulsifiers which may be advantageous are the following:

fatty alcohols having 8 to 30 carbon atoms, monoglycerol esters of saturated and/or unsaturated, branched and/or unbranched alkanecarboxylic acids having a chain length of 8 to 24, in particular 12-18, C atoms, diglycerol esters of saturated and/or unsaturated, branched and/or unbranched alkanecarboxylic acids having a chain length of 8 to 24, in particular 12-18, C atoms, monoglycerol ethers of saturated and/or unsaturated, branched and/or unbranched alcohols having a chain length of 8 to 24, in particular 12-18, C atoms, diglycerol ethers of saturated and/or unsaturated, branched and/or unbranched alcohols having a chain length of 8 to 24, in particular 12-18, C atoms, propylene glycol esters of saturated and/or unsaturated, branched and/or unbranched alkanecarboxylic acids having a chain length of 8 to 24, in particular 12-18, C atoms, and sorbitan esters of saturated and/or unsaturated, branched and/or unbranched alkanecarboxylic acids having a chain length of 8 to 24, in particular 12-18, C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG 30 dipolyhydroxystearate.

The compositions or preparations described are in various administration forms usually used for the application indicated. For example, a preparation as described above may, in particular, be in the form of a lotion or emulsion, such as in the form of a cream or milk (O/W, W/O, O/W/O, W/O/W), in the form of oily-alcoholic, oily-aqueous or aqueous-alcoholic gels or solutions, in the form of solid sticks or may be formulated as an aerosol.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, and other ingredients usually used in cosmetics.

The preservatives used are preferably approved preservatives which are listed in the Cosmetics Regulation, Annex 6, as positive list or also anti-microbial pigments, as described, for example, in WO 2004/0092283 or WO 2004/091567.

Suitable preservatives are therefore also alkyl esters of p-hydroxybenzoic acid, hydantoin derivatives, propionate salts or a multiplicity of ammonium compounds.

Very particularly preferred preservatives are methylparaben, propylparaben, imidazolidinylurea, sodium dehydroxyacetate or benzyl alcohol. Preservatives are employed in amounts between 0.5 and 2% by weight. The amount data are based on the total amount of the formulation.

Emollients or softeners are often incorporated into cosmetic preparations.

They are preferably employed in 0.5 to 50% by weight, preferably between and 30% by weight, based on the composition as a whole. In general, softeners can be classified in classes, such as, for example, the category of the esters, fatty acids or fatty alcohols, polyols, hydrocarbons and oils containing at least one amide structural unit.

Representative oils containing at least one amide structural unit together with their synthesis are described, in particular, in EP 1044676 and EP 0928608. A compound which is particularly preferably indicated is isopropyl N-lauroylsarcosinate, which is commercially available from Ajinomoto under the product name Eldew SL-205.

Of the esters, mono- or diesters can be selected. Examples in this respect are dibutyl adipate, diethyl sebacate, diisopropyl dimerate or dioctyl succinate. Branched fatty acid esters are, for example, 2-ethylhexyl myristate, isopropyl stearate or isostearyl palmitate. Tribasic esters are, for example, triisopropyl trilinoleate or trilauryl citrate. Straight-chain fatty acid esters are, for example, lauryl palmitate, myristyl lactate, oleyl erucate or stearyl oleate. Preferred esters are Coco-Caprylate/Caprate (=INCI name, these are esters of coconut fatty alcohols with saturated medium-chain fatty acids), propylene glycol myristyl ether acetate, diisopropyl adipate or cetyl octanoate.

Suitable fatty alcohols and acids are compounds which have 10 to 20 C atoms. Particularly preferred compounds are cetyl, myristyl, palmitic or stearic alcohol or acid.

Suitable polyols are linear or branched-chain alkylpolyhydroxyl compounds, for example propylene glycol, sorbitol or glycerol. However, it is also possible to employ polymeric polyols, for example polypropylene glycol or polyethylene glycol. Butylene glycol and propylene glycol are also particularly suitable compounds for enhancing the penetration capacity.

Examples of hydrocarbons as softeners are compounds which generally have 12 to 30 C atoms. Specific examples are arylalkyl benzoates, alkyl benzoates, mineral oils, Vaseline, squalenes or isoparaffins.

Further emollients or hydrophobicising agents are preferably C₁₂ to C₁₅ alkyl benzoates, dioctyl adipate, octyl stearate, octyldodecanol, hexyl laurate, octyldodecyl neopentanoate, cyclomethicone, dicaprylic ether, dimethicone, phenyltrimethicone, isopropyl myristate, caprylic/capric glycerides, propylene glycol dicaprylate/dicaprate or decyl oleate.

A further category of functional ingredients of cosmetic preparations are thickeners. Thickeners are generally employed in amounts between 0.1 and 20% by weight, preferably between 0.5 and 10% by weight, based on the total amount. Examples of these compounds are crosslinked polyacrylate materials, commercially available from B. F. Goodrich Company under the trade name Carbopol. It is also possible to use thickeners such as xanthan gum, carrageenan gum, gelatine gum, karaya gum, pectin gum or carob seed flour.

Under certain circumstances, it is possible for a compound to be both a thickener and also a softener. Examples thereof are silicone gums (kinematic viscosity>10 centistokes), esters, such as, for example, glycerol stearate, or cellulose derivatives, for example hydroxypropylcellulose.

The dispersant or solubiliser used can be an oil, wax or other lipid, a lower monoalcohol or lower polyol or mixtures thereof. Particularly preferred monoalcohols or polyols include ethanol, i-propanol, propylene glycol, glycerol and sorbitol.

A preferred embodiment of the invention is an emulsion in the form of a protective cream or milk which, in addition to the compound(s) of the formula I, comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The preparations described above may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The invention is explained in greater detail below with reference to examples. The invention can be carried out throughout the range claimed and is not restricted to the examples given here.

### EXAMPLES

### Example 1

### “Liquid Skin Model” Procedure as In-Vitro Tanning Model

A solution is prepared consisting of 94 ml of ethylene glycol, 6 ml of phosphate buffer pH=7, 146 mg of DL-lysine and 90 mg of DHA. The solids are initially introduced. The solution is subsequently transferred in equal proportions into two vessels A and B. Vessel A is tightly sealed so that supply of oxygen no longer takes place. Vessel B remains open. The two batches are stirred for 24 h.

Result: Batch A exhibits a surprisingly dark deep-brown coloration compared with batch B. By contrast, batch B merely has a yellowish colour. The calorimetric evaluation of the batches is tabulated below (L*a*b* colour measurement, a*=red-blue component; b*=yellow-green component; L*=light-dark component):

| | a* | b* | L* |
|---|---|---|---|
| Batch A | 0.9 | 0.9 | 0.5 |
| Batch B | −0.3 | 4.8 | 7.9 |
| Blank value (ethylene glycol/water = 96/4) | 0 | 0 | 100 |

### Example 2

### Pretreatment Preparation Comprising Sodium Sulfite

| Ingredient | % by weight |
|---|---|
| Phase A | |
| Glyceryl Stearate, Steareth-26, Ceteth-20, Stearyl Alcohol | 8.0 |
| Cetearyl Alcohol | 1.5 |
| Cetearyl Ethylhexanoate | 6.5 |
| Caprylic/Capric Triglyceride | 6.5 |
| Stearoxy Dimethicone | 1.2 |
| Dimethicone | 0.5 |
| Tocopheryl Acetate | 0.5 |
| Propylparaben | 0.05 |
| Phase B | |
| Propylene glycol | 3.0 |
| Methylparaben | 0.15 |
| Sodium bisulfite (Na₂S₂O₅) | 0.5 |
| Aqua (water) | to 100 |
| Perfume | 0.1 |

### Preparation Process:

Phases A and B are heated to 65-70° C. Phase B is subsequently added to phase A with stirring. After homogenisation, the mixture is allowed to cool to room temperature.

### Example 3

### Lotion (W/O) for Application to the Skin, Comprising DHA and Sodium Sulfite

| | | % by weight |
|---|---|---|
| A | Polyglyceryl 2-dipolyhydroxystearate | 5.0 |
| | Beeswax | 0.5 |
| | Zinc stearate | 0.5 |
| | Hexyl laurate | 9.0 |
| | Cetyl isononanoate | 6.0 |
| | Shea butter | 0.5 |
| | DL-α-tocopherol acetate | 1.0 |
| B | Glycerin | 5.0 |
| | Dihydroxyacetone | 2.0 |
| | Sodium sulfite | 0.5 |
| | Magnesium sulfate heptahydrate | 1.0 |
| | Preservatives | q.s. |
| | Water, demineralised | to 100 |

Alternatively to dihydroxyacetone, a mixture of dihydroxyacetone and troxerutin can be used, for example 3% by weight mixture comprising dihydroxyacetone and troxerutin in the ratio 2:1.

### Preparation

Phase A is warmed to 75° C. and phase B to 80° C. Phase B is slowly added to phase A with stirring. After homogenisation, the mixture is cooled with stirring. Perfumes can optionally be added at a temperature of 40° C.

The following preservatives are used:

0.05% of propyl 4-hydroxybenzoate
0.15% of methyl 4-hydroxybenzoate

### Example 4

### Lotion (W/O) for Application to the Skin, Comprising DHA, Erythrulose and Sodium Sulfite

| | | % by weight |
|---|---|---|
| A | Polyglyceryl 2-dipolyhydroxystearate | 5.0 |
| | Beeswax | 0.5 |
| | Zinc stearate | 0.5 |
| | Hexyl laurate | 9.0 |
| | Cetyl isononanoate | 6.0 |
| | Shea butter | 0.5 |
| B | Glycerin | 5.0 |
| | Dihydroxyacetone | 2.0 |
| | Erythrulose | 1.0 |
| | Sodium bisulfite | 0.5 |
| | Magnesium sulfate heptahydrate | 1.0 |
| | Preservatives | q.s. |
| | Water, demineralised | to 100 |

### Preparation

Phase A is warmed to 75° C. and phase B to 80° C. Phase B is slowly added to phase A with stirring. After homogenisation, the mixture is cooled with stirring. Perfumes can optionally be added at a temperature of 40° C.

The following preservatives are used:

0.05% of propyl 4-hydroxybenzoate
0.15% of methyl 4-hydroxybenzoate

### Example 5

### Self-Tanning Lotion, Inertised

| Ingredient | % |
|---|---|
| Phase A | |
| Cetearyl Alcohol, Cetearyl Glucoside | 4.0 |
| Sorbitanstearate | 1.5 |
| Cetearyl Alcohol | 2.0 |
| C12-13 Alkyl Lactate | 2.0 |
| Isohexadecane | 1.5 |
| Paraffinum Liquidum (Mineral Oil) | 3.5 |
| Cyclomethicone, Dimethicone Crosspolymer | 2.0 |
| Tocopheryl Acetate | 0.5 |
| Propylparaben | 0.05 |
| Phase B | |
| Glycerin | 2.0 |
| Aqua (Water) | to 100 |
| Sodium bisulfite | 0.5 |
| Methylparaben | 0.15 |
| Phase C | |
| Xanthan Gum | 0.2 |
| Phase D | |
| Dihydroxyacetone | 5.0 |
| Aqua, Alcohol denat., Lecithin, Glycerin, | 5.0 |
| Disodium Phosphate (=empty liposomes Probiol L05018) | |
| Aqua (Water) | 10.0 |
| Phase E | |
| Perfume | 0.2 |

Preparation: Phase B is inertised in an ultrasound bath until the oxygen content is below 5 mg/l. Phase B is optionally pretreated in a further inertisation step by introduction of nitrogen. Phases A and B are warmed separately to 75° C. Phase C is added to phase B. The combined phases B and C are added to phase A with stirring. The mixture is homogenised and cooled to 40° C., before phases D and E are added.

### Note:

It may be advisable also to subject phases A and C to inertisation.

### Example 6

### In-Vivo Test Experiment

An emulsion comprising 4% of DHA is applied to the left and right sub-arm of premarked, opposite fields (4.3 cm×4.3 cm). The application rate is 2 mg/cm². After about 10 minutes, the field on the left sub-arm is tightly wrapped with PE kitchen film in order to prevent oxygen exposure. After 5 hours, the PE film is removed. 22 hours after commencement of the experiment, it is observed that a significantly stronger tanning effect occurs in the case of oxygen reduction by covering with film.

1. Method for enhancing the tanning action of at least one self-tanner substance by reducing or eliminating the presence of oxygen.

2. Method according to claim 1, characterised in that the reduction or elimination of the presence of oxygen is carried out physically, chemically, biochemically or microbiologically.

3. Method according to claim 1, characterised in that the reduction or elimination of the oxygen content is achieved through the type of application of the at least one self-tanner substance.

4. Method according to claim 3, characterised in that the reduction or elimination of the oxygen content is achieved during application of the at least one self-tanner substance.

5. Method according to claim 4, characterised in that the application is carried out in a tanning reactor under a protective gas.

6. Method according to claim 3, characterised in that the reduction or elimination of the oxygen content is achieved by pretreatment of the skin to which the at least one self-tanner substance is applied.

7. Method according to claim 6, characterised in that the area to which the formulation comprising at least one self-tanner substance is applied is treated in advance with a formulation comprising at least one oxygen-binding or oxygen-withdrawing substance and/or at least one oxygen-consuming biochemical or microbiological component and/or at least one antioxidant.

8. Method according to claim 3, characterised in that the reduction or elimination of the oxygen content is achieved after application of the at least one self-tanner substance.

9. Method according to claim 8, characterised in that the treated area is covered by suitable materials.

10. Method according to claim 1, characterised in that the measures reduce the presence of oxygen to a partial pressure less than the atmospheric partial pressure of oxygen.

11. Method according to claim 1, characterised in that the reduction or elimination of the oxygen content is achieved through the type of formulation comprising the at least one self-tanner substance.

12. Method according to claim 11, characterised in that the preparation of the formulation is carried out with a reduction in the oxygen content.

13. Method according to claim 11, characterised in that the individual components of the formulation are degassed or inertised or the formulation is degassed or inertised.

14. Method according to claim 11, characterised in that the oxygen content in the aqueous component of the formulation is <10 mg/l.

15. Method according to claim 11, characterised in that the formulation comprises at least one oxygen-binding or oxygen-withdrawing component and/or an oxygen-consuming biochemical or microbiological component.

16. Method according to claim 7, characterised in that the oxygen-binding or oxygen-withdrawing component is selected from the group of the alkali metal, alkaline-earth metal or ammonium sulfites, alkali metal, alkaline-earth metal or ammonium hydrogensulfites, alkali metal, alkaline-earth metal or ammonium bisulfites, alkali metal, alkaline-earth metal or ammonium polysulfites or dialkylhydroxylamines.

17. Method according to claim 15, characterised in that the oxygen-consuming biochemical or microbiological component is selected from the group of superoxide dismutase, peroxidase and/or catalase.

18. Method according to claim 1, characterised in that the at least one self-tanner substance is selected from the group of glycerolaldehyde, hydroxymethylglyoxal, γ-dialdehyde, erythrulose, 6-aldo-D-fructose, ninhydrin, 5-hydroxy-1,4-naphthoquinone, 2-hydroxy-1,4-naphthoquinone, 1,3-dihydroxyacetone, dihydroxyacetone phosphate, glyceraldehyde phosphate or erythrose.

19. Inertised preparation comprising at least one self-tanner substance.

20. Preparation according to claim 19, characterised in that the at least one self-tanner substance is selected from the group of glycerolaldehyde, hydroxymethylglyoxal, γ-dialdehyde, erythrulose, 6-aldo-D-fructose, ninhydrin, 5-hydroxy-1,4-naphthoquinone, 2-hydroxy-1,4-naphthoquinone, 1,3-dihydroxyacetone, dihydroxyacetone phosphate, glyceraldehyde phosphate or erythrose.

21. Preparation according to claim 19, where the oxygen content in the aqueous component of the preparation is less than or equal to 10 mg/l.

22. Preparation comprising at least one self-tanner substance and at least one oxygen-consuming biochemical or microbiological component.

23. Preparation according to claim 22, characterised in that the at least one self-tanner substance is selected from the group of glycerolaldehyde, hydroxymethylglyoxal, γ-dialdehyde, erythrulose, 6-aldo-D-fructose, ninhydrin, 5-hydroxy-1,4-naphthoquinone, 2-hydroxy-1,4-naphthoquinone, 1,3-dihydroxyacetone, dihydroxyacetone phosphate, glyceraldehyde phosphate or erythrose.

24. Preparation according to claim 22 characterised in that the at least one oxygen-consuming biochemical or microbiological component is selected from the group of superoxide dismutase, peroxidase and/or catalase.

25. Kit comprising at least one preparation comprising at least one oxygen-binding or oxygen-withdrawing component and/or at least one oxygen-consuming biochemical or microbiological component and/or at least one antioxidant and at least one preparation comprising at least one self-tanner substance.

26. Kit according to claim 25, characterised in that the at least one oxygen-binding or oxygen-withdrawing component is selected from the group of the alkali metal, alkaline-earth metal or ammonium sulfites, alkali metal, alkaline-earth metal or ammonium hydrogensulfites, alkali metal, alkaline-earth metal or ammonium bisulfites, alkali metal, alkaline-earth metal or ammonium polysulfites or dialkylhydroxylamines.

27. Kit according to claim 25, characterised in that the at least one oxygen-consuming biochemical or microbiological component is selected from the group of superoxide dismutase, peroxidase and/or catalase.

28. Kit according to claim 25, characterised in that the at least one self-tanner substance is selected from the group of glycerolaldehyde, hydroxymethylglyoxal, γ-dialdehyde, erythrulose, 6-aldo-D-fructose, ninhydrin, 5-hydroxy-1,4-naphthoquinone, 2-hydroxy-1,4-naphthoquinone, 1,3-dihydroxyacetone, dihydroxyacetone phosphate, glyceraldehyde phosphate or erythrose.

29. Kit comprising a preparation comprising at least one self-tanner substance and a film whose oxygen permeability has a value of less than 1000 cm³/(m²*bar*d).

30. Kit according to claim 29, characterised in that the at least one self-tanner substance is selected from the group of glycerolaldehyde, hydroxymethylglyoxal, γ-dialdehyde, erythrulose, 6-aldo-D-fructose, ninhydrin, 5-hydroxy-1,4-naphthoquinone, 2-hydroxy-1,4-naphthoquinone, 1,3-dihydroxyacetone, dihydroxyacetone phosphate, glyceraldehyde phosphate or erythrose.
